(19) United States
(12) Patent Application Publication
(10) Pub. No.: US 2025/0082904 A1
(43) Pub. Date: Mar. 13, 2025

(54) Secondary Securement Device for Vascular Access Device

(71) Applicant: Becton, Dickinson and Company, Franklin Lakes, NJ (US)

(72) Inventors: Atharva Shetye, Bangalore (IN); Divik Bhargava, Meerut (IN); Rahul Malviya, Salmon Arm (CA); Srimannarayana Myla, Pune (IN); Shishir Prasad, Ramsey, NJ (US)

(21) Appl. No.: 18/571,873

(22) PCT Filed: Dec. 20, 2022
(86) PCT No.: PCT/US2022/053451

§ 371 Date: Dec. 19, 2023

(51) Int. Cl. A61M 25/02

(52) U.S. Cl. CPC A61M 25/02

(57) ABSTRACT

A securement device for a vascular access device includes a base including a first end, a second end, a first side, a second side, a top surface, and a bottom surface positioned opposite the top surface, with the bottom surface configured to be in contact with a patient's skin surface, a first securing member including a first end connected to the base and a second end opposite from the first end, the second end moveable relative to the base. The first securing member defines a first opening configured to receive a first portion of the vascular access device, and a second securing member includes a first end connected to the base and a second end positioned opposite the first end, the second end moveable relative to the base and the second securing member defining a second opening configured to receive a second portion of the vascular access device.

### BACKGROUND OF THE INVENTION

### Field of the Invention

The present disclosure relates to a secondary securement device for a vascular access device.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal. Common types of VADs are an over-the-needle peripheral intravenous catheter (PIVC) or central venous catheter (CVC). A variety of solutions exist for securing VADs and maintaining the insertion site; however, clinicians are often left with limited solutions to manage the portions of the lumen that extend outside of the body. These loose extensions can create opportunities for the lumens to be pulled, potentially compromising the catheter position or insertion site integrity. Tape and textile dressings are sometimes utilized, but can be cumbersome when the line needs to be accessed. In addition, while a line is being accessed, an un-capped line is at risk for touch contamination as the clinician often needs to perform multiple steps.

### SUMMARY OF THE INVENTION

In one aspect or embodiment, a secondary securement device for a vascular access device includes a base including a first end, a second end positioned opposite the first end, a first side, a second side positioned opposite the first side, a top surface, and a bottom surface positioned opposite the top surface, with the bottom surface configured to be in contact with a patient's skin surface, a first securing member including a first end connected to the base and a second end positioned opposite from the first end, with the second end moveable relative to the base and the first securing member defining a first opening configured to receive a first portion of the vascular access device, a second securing member including a first end connected to the base and a second end positioned opposite from the first end, with the second end moveable relative to the base and the second securing member defining a second opening configured to receive a second portion of the vascular access device.

The first opening may have a first diameter and the second opening has a second diameter larger than the first diameter. The first portion of the vascular access device may be tubing, and the second portion of the vascular access device may be a connector positioned on the tubing. The first and second securing members may each have a first position where the base and the first and second securing members are positioned coplanar, and a second position where the first and second securing members are positioned at a non-zero angle relative to the base. The non-zero angle may be 45-135 degrees. The first opening may include a spherical portion and an elongated portion, with the elongated portion extending from the second end of the first securing member to the spherical portion. The second opening may include a spherical portion and an elongated portion, with the elongated portion extending from the second end of the second securing member to the spherical portion. The first and second securing members may be defined by a plurality of slits in the base. The first and second securing members may be moveable relative to the base via a living hinge. The bottom surface of the base may include an adhesive surface. The secondary securement device may further include a third securing member having a first end connected to the base and a second end positioned opposite from the first end, with the second end moveable relative to the base and the third securing member defining a third opening configured to receive a third portion of the vascular access device.

In a further aspect or embodiment, a method of using the secondary securement device according to any of the above aspects or embodiments includes: positioning tubing of the vascular access device within the first opening of the first securing member to secure the tubing; and positioning a connector of the vascular access device within the second opening of the second securing member to secure the connector.

The method may further include: pushing the tubing of the vascular access device past the elongated portion of the first opening; and pushing the connector of the vascular access device past the elongated portion of the second opening.

### BRIEF DESCRIPTION OF THE DRAWINGS

FIG. 1 is a perspective view of a secondary securement device for a vascular access device according to one aspect or embodiment of the present application;

FIG. 2 is a top view of the secondary securement device of FIG. 1;

FIG. 3 is a perspective view of the secondary securement device of FIG. 1, showing a use position of the secondary securement device;

FIG. 4 is a perspective view of the secondary securement device of FIG. 1, showing the secondary securement device secured to a skin surface of a patient; and

FIG. 5 is a perspective view of the secondary securement device of FIG. 1, showing the secondary securement device secured to a skin surface of a patient with a vascular access device secured using the secondary securement device.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

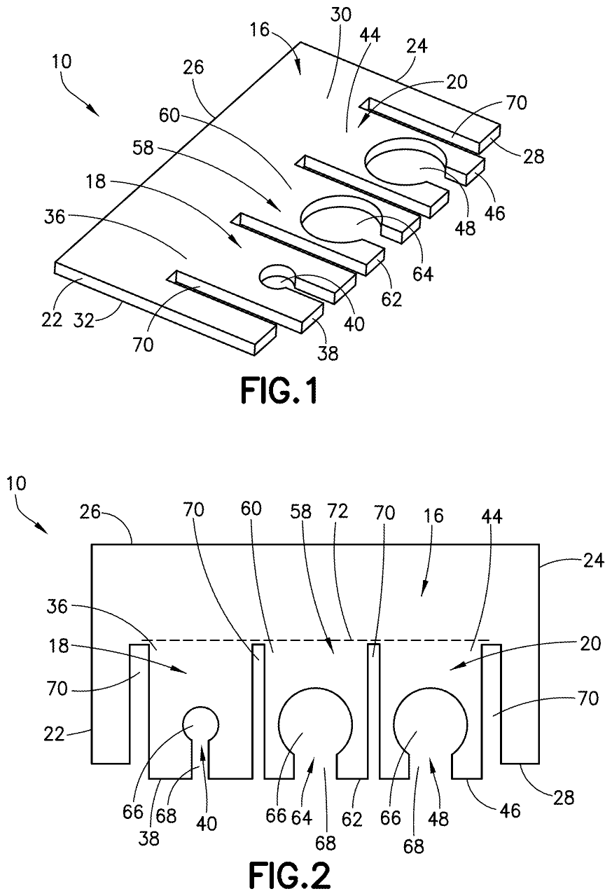

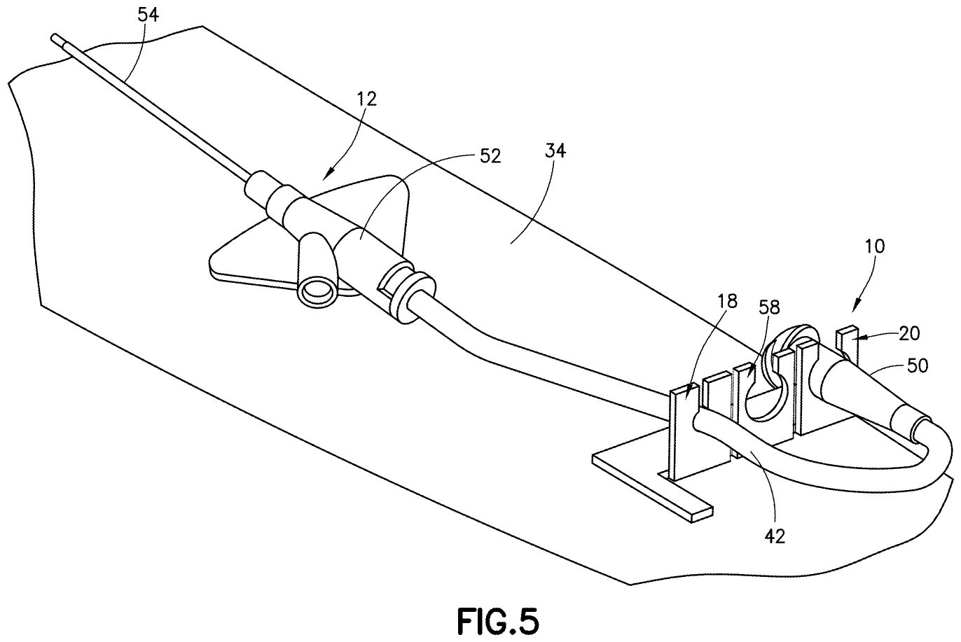

Referring to FIGS. 1-5, in one aspect or embodiment, a secondary securement device 10 for a vascular access device 12 includes a base 16, a first securing member 18, and a second securing member 20. The base 16 includes a first end 22, a second end 24 positioned opposite the first end 22, a first side 26, a second side 28 positioned opposite the first side 26, a top surface 30, and a bottom surface 32 positioned opposite the top surface 30. The bottom surface 32 is configured to be in contact with a patient's skin surface 34. The first securing member 18 includes a first end 36 connected to the base 16 and a second end 38 positioned opposite from the first end 36, with the second end 38 moveable relative to the base 16. The first securing member 18 defines a first opening 40 configured to receive a first portion 42 of the vascular access device 12. The second securing member 20 includes a first end 44 connected to the base 16 and a second end 46 positioned opposite from the first end 44, with the second end 46 moveable relative to the base 16. The second securing member 20 defines a second opening 48 configured to receive a second portion 50 of the vascular access device 12. As shown in FIG. 5, in some aspects or embodiments, the first portion 42 of the vascular access device 12 is tubing, and the second portion 50 of the vascular access device 12 is a connector positioned on the tubing 42.

As discussed in additional detail below, the secondary securement device 10 is configured to secure the tubing 42 of the vascular access device 12 to prevent accidental pull forces from transmitting to the vascular access device 12 and placed catheter. The vascular access device 12 may be a peripheral intravenous catheter or central venous catheter, although the secondary securement device 10 may be utilized in connection with other medical devices. In some aspects or embodiments, the vascular access device 12 includes a catheter adapter 52, a catheter 54, the tubing 42, and the connector 50 positioned at a proximal end of the tubing 42.

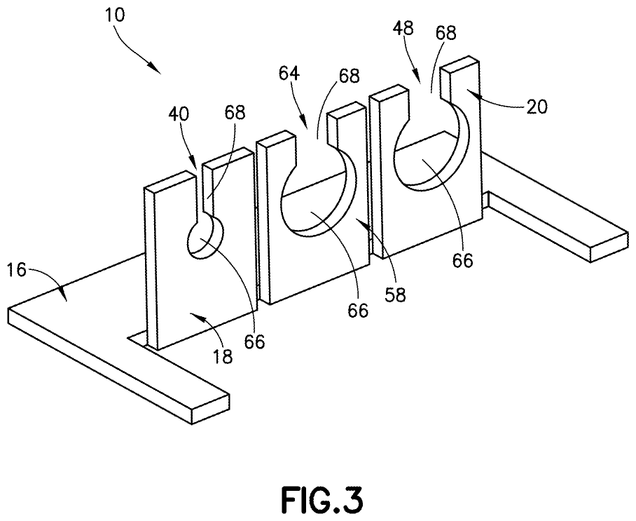

Referring to FIGS. 1-3, in some aspects or embodiments, the secondary securement device 10 includes a third securing member 58 having a first end 60 connected to the base 16 and a second end 62 positioned opposite from the first end 60, with the second end 62 moveable relative to the base 16. The third securing member 58 defines a third opening 64 configured to receive a third portion of the vascular access device 12, such as a further connector (not shown), further tubing (not shown), or another portion of the tubing 42. Although three securing members 18, 20, 58 are shown, one or more securing members may be provided. The first opening 40 has a first diameter and the second opening 48 has a second diameter larger than the first diameter. The third opening 64 is the same diameter as the second opening 48, although other suitable size and arrangements may be utilized. As discussed above, the first opening 40 is sized and configured to receive and secure the tubing 42 of the vascular access device 12 and the second opening 48 is sized and configured to receive and secure the connector 50 of the vascular access device 12. The number of securing members 18, 20, 58 and openings 40, 48, 64 may be scaled to accommodate the multiple lumens for multi-lumen catheters. The first, second, and third securing members 18, 20, 58 each have a first position (FIG. 1) where the base 16 and the securing members 18, 20, 58 are positioned coplanar and a second position (FIG. 3) where the securing members 18, 20, 58 are positioned at a non-zero angle relative to the base 16. In some aspects or embodiments, the non-zero angle is 45-135 degrees. The movement of the first, second, and third securing members 18, 20, 58 is configured to act as damper when forces are applied to a hub end of the vascular access device 12 thereby reducing the impact of the forces or pull to the placed catheter of the vascular access device 12.

Referring to FIG. 2, the first, second, and third openings 40, 48, 64 each include a spherical portion 66 and an elongated portion 68, with the elongated portion 68 extending from the second end 38, 46, 62 of the securing members 18, 20, 58 to the spherical portion 66. The tubing 42 or connector 50 of the vascular access device 12 is configured to engage the elongated portion 68 of the openings 40, 48, 64 and bias the openings 40, 48, 64 radially outward as the tubing 42 or connector 50 is positioned within the spherical portion 66 of the openings 40, 48, 64. The elongated portion 68 of the openings 40, 48, 64 is configured to retain the tubing 42 or connector 50 within the spherical portion 66 of the openings 40, 48, 64. The first, second, and third securing members 18, 20, 58 are defined by a plurality of slits 70 in the base 16. The first, second, and third securing members 18, 20, 58 are moveable relative to the base 16 via a living hinge 72, although other suitable arrangements may be utilized. The bottom surface 32 of the base 16 includes an adhesive surface. Although not shown, the adhesive surface may include a liner that is configured to be peeled or removed prior to placing the secondary securement device 10. The secondary securement device 10 may be formed as a single piece of material, such as by die-cutting, molding, or other suitable process.

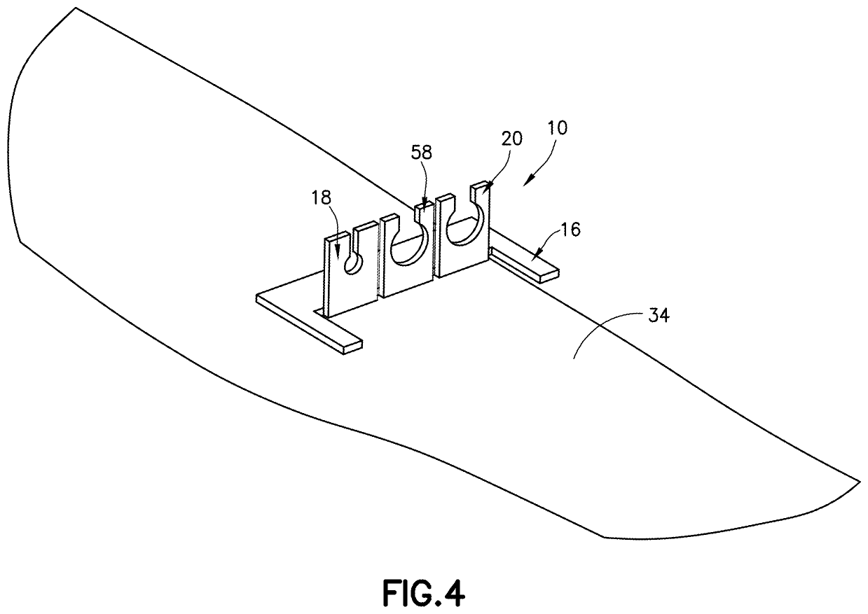

Referring to FIGS. 4 and 5, in one aspect or embodiment, a method of using the secondary securement device 10 includes: positioning the tubing 42 of the vascular access device 12 within the first opening 40 of the first securing member 18 to secure the tubing 42; and positioning the connector 50 of the vascular access device 12 within the second opening 48 of the second securing member 20 to secure the connector 50. As shown in FIG. 4, the secondary securement device 10 may be first positioned on the skin surface 34 of the patient by placing the adhesive surface of the bottom surface 32 of the base 16 onto the skin surface 34. The securing members 18, 20, 58 may initially be in the first position when placed on the skin surface 34 and subsequently moved to the second position shown in FIG. 4, although the securing members 18, 20, 58 may also be placed in the second position prior to placement on the skin surface 34. The method may further include pushing the tubing 42 of the vascular access device 12 past the elongated portion 68 of the first opening 40; and pushing the connector 50 of the vascular access device 12 past the elongated portion 68 of the second opening 48. The tubing 42 of the vascular access device 12 may be formed in to a J-shape or inverse J-shape, although other suitable configurations may be utilized.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

1. A secondary securement device for a vascular access device comprising:
a base including a first end, a second end positioned opposite the first end, a first side, a second side positioned opposite the first side, a top surface, and a bottom surface positioned opposite the top surface, the bottom surface configured to be in contact with a patient's skin surface;
a first securing member including a first end connected to the base and a second end positioned opposite from the first end, the second end moveable relative to the base, the first securing member defining a first opening configured to receive a first portion of the vascular access device;
a second securing member including a first end connected to the base and a second end positioned opposite from the first end, the second end moveable relative to the base, the second securing member defining a second opening configured to receive a second portion of the vascular access device.

2. The secondary securement device of claim 1, the first opening has a first diameter and the second opening has a second diameter larger than the first diameter.

3. The secondary securement device of claim 1, wherein the first portion of the vascular access device comprises tubing, and wherein the second portion of the vascular access device comprises a connector positioned on the tubing.

4. The secondary securement device of claim 1, wherein the first and second securing members each have a first position where the base and the first and second securing members are positioned coplanar and a second position where the first and second securing members are positioned at a non-zero angle relative to the base.

5. The secondary securement device of claim 4, wherein the non-zero angle is 45-135 degrees.

6. The secondary securement device of claim 1, wherein the first opening comprises a spherical portion and an elongated portion, the elongated portion extending from the second end of the first securing member to the spherical portion.

7. The secondary securement device of claim 6, wherein the second opening comprises a spherical portion and an elongated portion, the elongated portion extending from the second end of the second securing member to the spherical portion.

8. The secondary securement device of claim 7, wherein the first and second securing members are defined by a plurality of slits in the base.

9. The secondary securement device of claim 1, wherein the first and second securing members are moveable relative to the base via a living hinge.

10. The secondary securement device of claim 1, wherein the bottom surface of the base comprises an adhesive surface.

11. The secondary securement device of claim 1, further comprising a third securing member including a first end connected to the base and a second end positioned opposite from the first end, the second end moveable relative to the base, the third securing member defining a third opening configured to receive a third portion of the vascular access device.

12. A method of using the secondary securement device of claim 1, the method comprising:
positioning tubing of the vascular access device within the first opening of the first securing member to secure the tubing; and
positioning a connector of the vascular access device within the second opening of the second securing member to secure the connector.

13. The method of claim 12, wherein the first opening comprises a spherical portion and an elongated portion, the elongated portion extending from the second end of the first securing member to the spherical portion, and wherein the second opening comprises a spherical portion and an elongated portion, the elongated portion extending from the second end of the second securing member to the spherical portion, the method further comprising:
pushing the tubing of the vascular access device past the elongated portion of the first opening; and
pushing the connector of the vascular access device past the elongated portion of the second opening.
